(19) United States
(12) Patent Application Publication
(10) Pub. No.: US 2017/0231552 A1
(43) Pub. Date: Aug. 17, 2017

(54) Joint Degeneration Grading Method and System

(71) Applicant: Christiaan Rudolf Oosthuizen, Northcliff (ZA)

(72) Inventor: Christiaan Rudolf Oosthuizen, Northcliff (ZA)

(21) Appl. No.: 15/434,303

(22) Filed: Feb. 16, 2017

(30) Foreign Application Priority Data
Feb. 16, 2016 (GB) 1602708.8

(51) Int. Cl. A61B 5/00

(52) U.S. Cl. CPC A61B 5/4585; A61B 5/4528; A61B 5/4533; A61B 5/4842; A61F 2/38

(57) ABSTRACT

This disclosure relates to a method and system for diagnosing the extent of degeneration of a knee joint, and determining a suitable prosthesis for use in knee arthroplasty as indicated by the extent of joint degeneration. According to the disclosure there is provided a method of determining a corrective surgical procedure for the treatment of degeneration of a joint, including the steps of comparing a set of radiological images, and in particular a set of x-ray images of the joint, to a set of degenerative joint conditions to determine a diagnosis; and correlating the diagnosis with a grading system which prescribes a corrective surgical procedure. The prescribed procedure may be a total knee arthroplasty, a partial knee arthroplasty, a patellofemoral arthroplasty or an anterior cruciate ligament reconstruction.

### RELATED APPLICATION

This application claims priority to United Kingdom Patent Application Serial No. 1602708.8, filed Feb. 16, 2016, which is incorporated by reference herein in its entirety.

### FIELD

This disclosure relates to a method for diagnosing the extent of degeneration of a joint and determining a suitable prosthesis for use in joint arthroplasty as indicated by the extent of joint degeneration of the joint, and more particularly, to a knee joint and knee arthroplasty.

### BACKGROUND

Damage to a knee joint, and specifically to areas of contact wherein relative motion during articulation occur, often causes discomfort or pain, and limits joint mobility. Damage is usually caused by, inter alia, genetic predisposition, trauma and/or aging, of which, osteoarthritis during ageing is the most common single cause.

Damage to the knee joint takes various forms, including chondromalacia, attrition of articular cartilage and/or that of the meniscal cartilage. Non-invasive treatment known to be used in less severe presentations include the administration of NSAIDS, cortisone injections, hyaluronic acid injections and arthroscopic debridement. Knee arthroplasty is common in the field of orthopaedic surgery as treatment for aggravated damage or injury to a knee joint.

In treating these more severe cases, two main techniques of treatment currently exist, namely the traditionally used total knee arthroplasty (TKA) and, more recently, partial knee arthroplasty (PKA), also termed unicompartmental knee arthroplasty (UKA). In both cases, typical prosthetic elements include femoral connecting and tibial connecting components, as well as a meniscal component sandwiched thereinbetween. The bone connecting components are typically manufactured from a high strength and high hardness metal, having highly polished exterior surfaces and roughened interior surfaces, whereas the meniscal component is typically manufactured from ultra high molecular weight polyethylene (UHMWPE). The interface between the polished exterior surfaces of the bone connecting components and the meniscal component promotes reduced friction during relative motion, whilst facilitating effective load transfer. The roughened interior surfaces on the other hand, promote attachment to bony tissue.

A couple of differences exist between TKAs and PKAs. Firstly, as the name suggests, a PKA can be used in one of three knee compartments, being the medial or the lateral tibiofemoral compartment, or the patellofemoral compartment, and thus only one compartments' femoral surface and tibial plateau or patella is resurfaced, while the other compartment's surfaces remain unaltered. A TKA's components extend over both compartments of both the distal femur and the proximal tibia, while a singular meniscal component also extends over both lateral and medial compartments of the knee.

Secondly, the range of motion of a TKA is restricted when compared to a PKA. PKAs retain constitutional alignment and knee function by restoring damaged features (i.e. condylar resurfacing), whilst substantially preserving ligamenture and normal knee anatomy, whereas TKAs provide a mechanical substitute for ligamenture and normal knee anatomy, thereby providing a range of controlled motion for the knee, with a view of artificially simulating normal knee function, but without generally achieving constitutional alignment. “Constitutional alignment” in the field of the present application refers to the dynamics, geometry and functioning of the knee of an actual individual patient. It should be borne in mind that certain misalignments may naturally occur in different patients. An individual's anatomy (especially hips and ankles), is generally adapted to these misalignments in the knee. A PKA procedure is aimed at retaining constitutional alignment of the knee, thus allowing knee, ankle and hip joints to retain its normal alignment and functioning within the leg of a specific patient. This essentially means that resultant complications in other body parts are avoided or at least minimised insofar as severity and prevalence thereof are concerned.

A TKA procedure, wherein a mechanical substitute for ligamenture and normal knee anatomy is provided and wherein constitutional alignment is not necessarily retained, forces the patient's knee to conform to “normal” knee movements as dictated by the prostheses, often making resultant complications in other body parts inevitable. TKAs generally limit relative motion between the femoral and meniscal components to rotation about a single, predetermined axis. The relative motion between the meniscal and the tibial components is severely limited and in most cases only minimal, single axis rotation and small degrees of translation is allowed. This range of controlled motion provided by a TKA, being non-constitutional motion, is what provides stability to the joint after natural means of stability, in the form of ligaments and tendons, had been sacrificed.

Load distribution within the knee is asymmetrical with the medial compartment supporting a larger fraction thereof. In osteoarthritic cases, the medial compartment's load bearing surfaces are thus the first to be degenerated, which may lead to varus (bow-legged) deformity. In cases where the lateral compartment wears more aggressively than the medial, valgus (knock-kneed) deformity occurs.

From the first instance, a surgeon always attempts to limit the extent of the invasiveness of the procedure by means of following a minimally invasive approach. Retaining constitutional alignment should always be preferred, whilst limiting the range of motion should be avoided. This is, obviously, not always possible, and thorough evaluation should accompany a decision between a TKA and a PKA. It is however, often found that only a single compartment of the knee is affected and therefore replacing all compartments will be unnecessarily invasive and limiting. From the aforementioned it is obvious that PKAs should be used where clinical evaluation supports it, rather than the more onerous and invasive TKA alternative.

Recent developments in the field of PKAs have increased the life expectancy of a PKA to up to 20 years and possibly even beyond, levelling it with that of TKAs. Further to this, minimally invasive revision of a PKA is now possible without affecting any of the ligaments, tendons, bony tissue or natural anatomy of a patient, resulting in significantly reduced post-operative recovery times. The use of a PKA in the first and subsequent instances can significantly delay, or even sometimes eliminate the need for a TKA. In recent times, the use of PKAs has seen a significant increase in popularity, but some surgeons still remain hesitant to recommend or perform these procedures.

In certain instances where both compartments of the knee is damaged or injured, but where ligaments retain stability, PKAs may sometimes be used in both compartments simultaneously, resulting in the same advantages as mentioned above. In severe cases however, the use of a TKA may be inevitable.

Some complications do however still occur when opting for a PKA, with dislocation of the meniscal component and aseptic loosening of the bone connecting surfaces being the most prevalent. Such complications often necessitate further surgery wherein the PKA is removed, and a TKA is performed.

With the improvements introduced herein, it is believed that the popularity of PKAs will further improve. In the USA, only about 9% of knee arthroplasties comprise PKAs. It is however believed that more than 50% of all damaged or injured knees in need of arthroplasty may in fact be suitable candidates for PKAs. Surgeons are, however, currently still reluctant to implement PKAs over TKAs due to difficulties experienced during the diagnosis phase of the extent of the damage or injury to the patient's knee and due to the inherent dangers of using the wrong option, including, but not limited to patient discomfort, increased post operative recovery times and decreased joint mobility. It should be borne in mind that once a TKA has been implemented, it can never be replaced by a PKA, while on the other hand, utilising a PKA in the first instance does not suffer the same fate. International patent application no. PCT/IB2012/055192 in the name of Oosthuizen for instance provides an alternative wherein an existing PKA can be revised by replacing the meniscal component by means of a minimally invasive anterior incision in the knee, which extends the lifespan of the PKA, and delays or prevents future TKAs which are significantly more invasive.

One of the challenges faced by orthopaedic surgeons is the lack of a uniform set of guidelines for identifying the competence of using a PKA instead of a TKA. Presently, reliance is made on a full medical history supplemented by clinical evaluation, which is often laborious, while still not providing a set criteria for consideration. In particular, the use of a PKA, especially in borderline cases, is ultimately a subjective determination on the part of the surgeon. This may lead to disuniformity across practiced treatment regimes and thus perpetuate doubt among practicing surgeons. This, can in turn, result in TKAs being favoured over PKAs, even where the latter, less invasive alternative would have been suitable, and which, if properly evaluated, would not have deprived the patient of the advantages of constitutional alignment and the general advantages of PKAs as aforementioned. The opposite, where a PKA was never suitable but erroneously utilised, is also possible, with immediate revision surgery and associated consequences an inevitable result.

From the above it is clear that a need exists for a system or method supplementing the current known evaluation method utilising full medical history and clinical evaluation on which appropriate treatment for a suitable patient is selected.

Accordingly, there is a need in the art to provide a method with which the abovementioned disadvantages can at least partially be overcome or alleviated by providing a grading system correlating with the degree of degeneration of a joint, with the grading system prescribing a corrective surgical procedure based on the extent of joint degeneration.

### SUMMARY

According to the disclosure there is provided a method of determining a corrective surgical procedure for the treatment of degeneration of a joint, including the steps of:
comparing a radiological image of the joint to a set of degenerative joint conditions to determine a diagnosis; and
correlating the diagnosis with a grading system which prescribes a corrective surgical procedure.

Further according to the disclosure, the joint may be a knee joint comprising a weight bearing joint, consisting of two opposing tibiofemoral (TF) compartments, and a patellofemoral (PF) joint.

Still further according to the disclosure, the set of degenerative joint conditions may comprise TF degeneration, PF degeneration, patellar grooving, subluxation of the weight bearing joint and ligament instability, and in particular instability of the anterior cruciate ligament (ACL).

Yet further according to the disclosure, the corrective surgical procedure may be a total knee arthroplasty (TKA), a partial knee arthroplasty (PKA), a patellofemoral arthroplasty (PFA) or an ACL reconstruction.

Even further according to the disclosure, the grading system may comprise a plurality of grades according to which the extent of degeneration of the knee joint may be determined, and whereby the corrective surgical procedure may subsequently be indicated. The grading system may comprise four grades, wherein the first may either be defined by degeneration confined to either of the TF compartments and wherein a PKA may be indicated, or by degeneration confined to the PF joint and wherein a PFA may be indicated. A second grade may be defined by degeneration confined to either of the TF compartments, resulting in subluxation across the weight bearing joint, and wherein a PKA may be indicated. A third grade may be defined by degeneration confined to either of the TF compartments with concomitant factors present. The concomitant factors may either take the form of ACL instability or a previous ACL reconstruction, wherein ACL reconstruction and a PKA may be indicated or lateral facet PF grooving, wherein a PFA and a PKA may be indicated. A fourth grade may be defined by degeneration present in both TF compartments. Here the stability of the ligaments may play a determining role in indicating a corrective surgical procedure. Firstly, if the ligaments are stable, the indicated surgical procedure may be either a bi-condylar PKA or a bi-cruciate retaining TKA, while, secondly, if the ligaments are unstable, resulting in subluxation across the weight bearing joint, a TKA may be indicated. In all of the above grades where degeneration is confined to a single TF compartment, a wedge deformity visualized on a stress view in the TF compartment unaffected by degeneration may be a contra-indication to the indicated corrective surgical procedure.

Yet further according to the disclosure, the determination of the grade of joint degeneration may be made by analyzing a radiological image of a knee joint of a patient, thereby identifying stages of joint degeneration, and comparing the identified stages of joint degeneration with the set of degenerative joint conditions.

The radiological image may be an x-ray image and may comprise an x-ray of a standing knee taken antero-posteriorly, a lateral view, a “stress-view” in 20° flexion, in 15° (medial) or 45° (lateral) postero-antero view (Rosenberg view) and a skyline view of the patella.

### BRIEF DESCRIPTION OF THE FIGURES

The disclosure will now be described in greater detail, by way of non-limiting example, with reference to the following drawings, in which:

FIG. 1 shows a schematic anterior view of a normal human knee;

FIG. 2 shows a schematic anterior view of a human knee, wherein bone-on-bone contact is present in the medial tibiofemoral (TF) compartment;

FIG. 3 shows a schematic skyline view of a normal human knee;

FIG. 4 shows a schematic skyline view of a human knee, wherein bone-on-bone contact between the femur and patella is present;

FIG. 5 shows a schematic anterior view of a human knee, wherein subluxation as well as bone-on-bone contact in the medial TF compartment is present;

FIG. 6 shows a schematic lateral view of a normal knee wherein anterior cruciate ligament (ACL) instability is present as the tibia subluxates to anterior;

FIG. 7 shows a schematic anterior view of a human knee, wherein bone-on-bone contact is present in both TF compartments due to degeneration;

FIG. 8 shows a schematic anterior view of a human knee, wherein subluxation as well as bone-on-bone contact is present in both TF compartments, due to TF degeneration and ACL instability;

FIG. 9 shows an anterior-posterior stress view of the knee, where the lateral TF compartment is unaffected; and

FIG. 10 shows an anterior-posterior stress view of the knee, where the medial TF compartment shows degeneration.

### DESCRIPTION

In the accompanying drawings, like numerals refer to like parts, unless otherwise indicated.

According to a embodiment of the present disclosure, there is provided a method of evaluating the severity or extent of degeneration to a knee joint of a human subject, and to prescribe a suitable prosthesis, if required, for use in joint arthroplasty. The method is proposed to be used in conjunction with clinical examination and adherence to previous contraindications as proposed by expected norms and standards in the art.

The method of evaluating the extent of degeneration of three compartments of the knee (i.e. medial and lateral tibiofemoral (TF) compartments and the patellofemoral PF compartment), in accordance with the present disclosure, is specifically practiced on a set of radiographic images of the knee joint of a specific patient. This enables a surgeon to evaluate the extent of degeneration to the knee joint in vitro. Obviously, certain advantages, and in particular the fact that the evaluation of the extent of degeneration can be done by an expert in the field without having to consult directly with the patient, arise when performing the evaluation in vitro. Further to this point, a surgeon about to perform an indicated type of arthroplasty, especially in cases where clinical evaluation indicated a borderline case, can thus with ease obtain a second opinion from a peer.

The set of radiographic images used during the evaluation of the degeneration of the knee joint includes anyone or a combination of x-ray images of a standing knee taken antero-posteriorly, a lateral view, a “stress-view” in 20° flexion, in 15° (medial) or 45° (lateral) postero-antero view (Rosenberg view) and a skyline view of the patella.

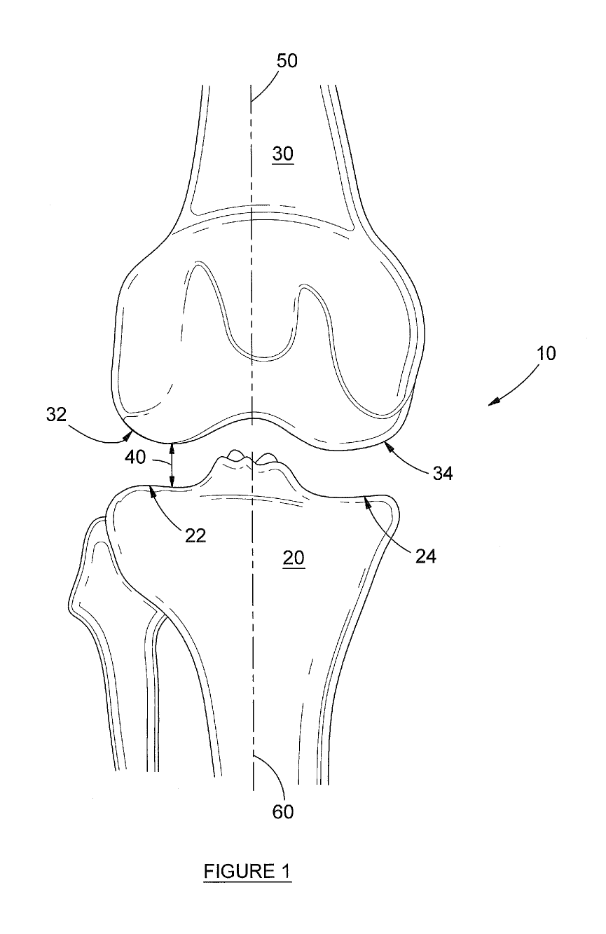

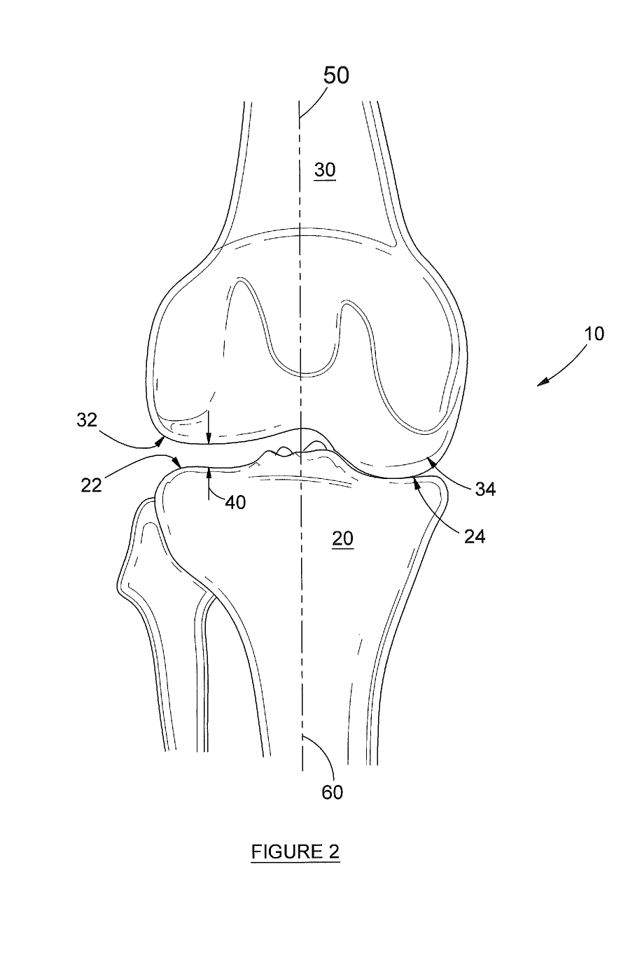

Turning now to FIGS. 1 and 2, reference numeral 10 generally refers to a human knee joint. A knee joint (10) generally allows articulation between a femur (30) and a tibia (20). The knee allows flexion, extension, and medial and lateral rotation of the tibia (20) relative to the femur (30). Apart from the abovementioned articulation, the knee joint also facilitates load transfer from the femur to the tibia. Load transfer is effected through interacting surfaces on both the distal femur and the proximal tibia. A typical knee (10) has two tibiofemoral (TF) or weight-bearing compartments, namely a lateral and a medial compartment. The lateral femoral condyle (32) interacts with the lateral tibial plateau (22), to collectively form the lateral TF compartment, while the medial femoral condyle (34) interacts with the medial tibial plateau (24), to collectively form the medial TF compartment. A joint spacing (40) is defined between the femur (30) and the tibia (20). The depth of the joint spacing (40) depends on several factors, namely the presence of a medial and lateral meniscus (not shown), cartilage on the femoral condyles and tibial plateau (not shown), as well as the presence and integrity of ligaments (not shown). It is the meniscus and the chondral cartilage that prevents bone-on-bone contact between the femoral condyles and the tibial plateau, effects load transfer from the femur (30) to the tibia (20) and reduces friction between contacting surfaces during articulation. The ligaments (not shown) provide stability to the knee joint (10). The alignment of a normal knee joint (10) is such that the femoral centre line (50) and the tibial centre line (60) meet within the joint space (40).

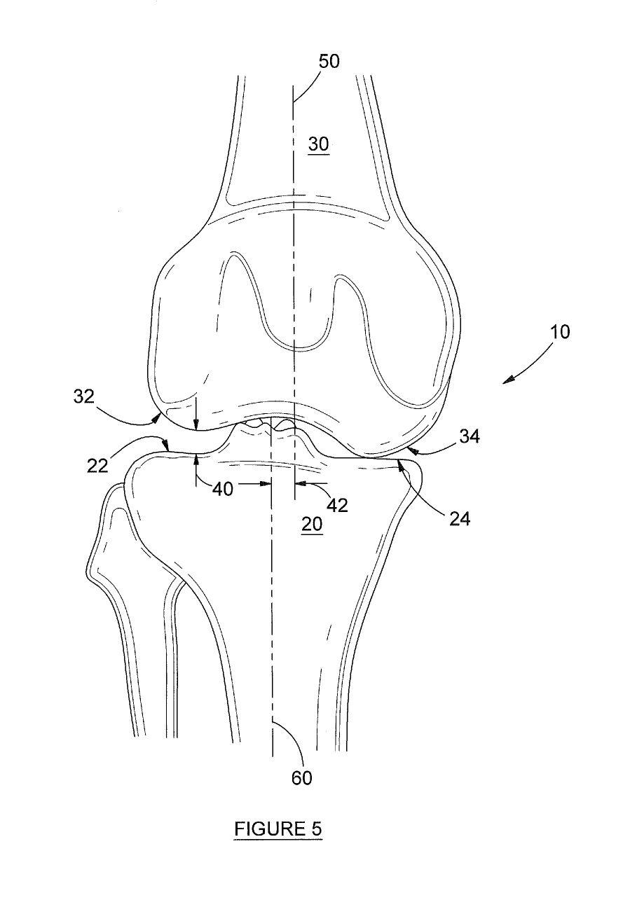

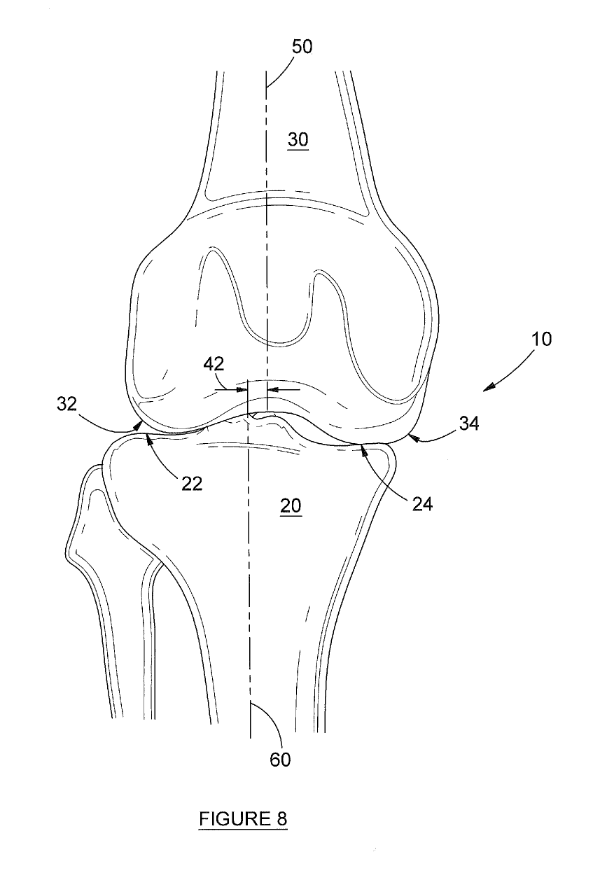

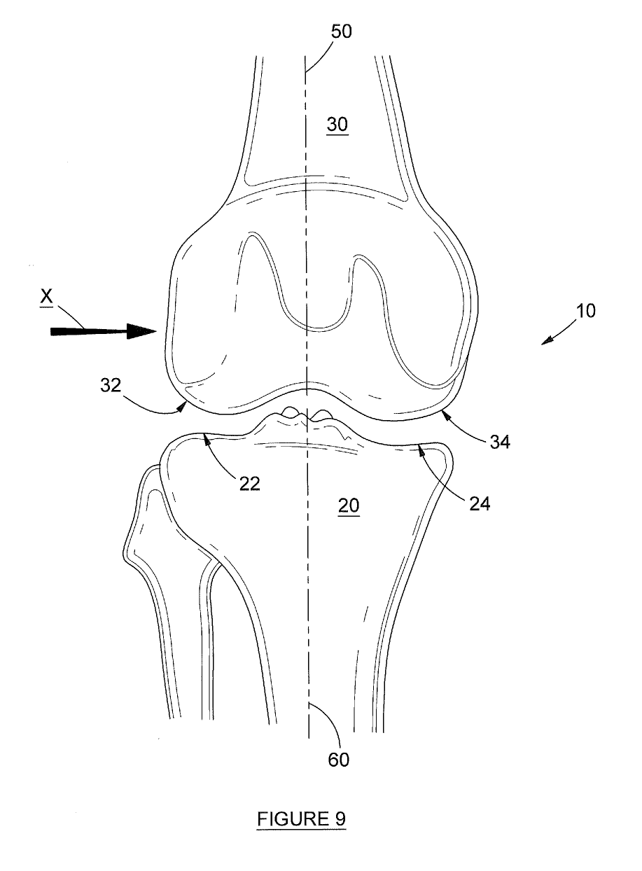

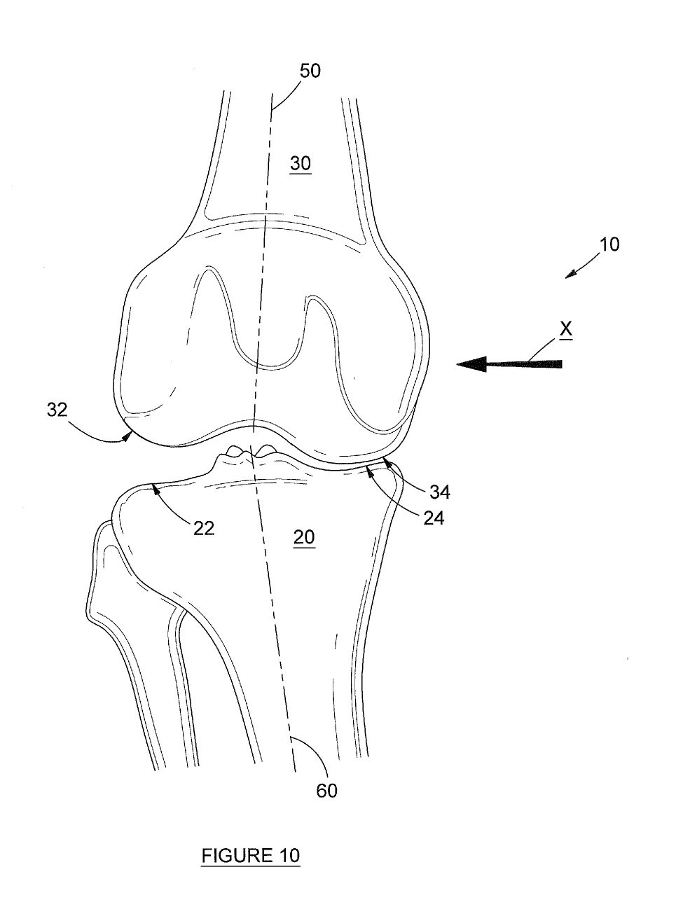

Damage or injury to a knee usually perpetuates from degeneration of the menisci and chondral cartilage, ultimately resulting in bone-on-bone contact between the femoral condyles (32, 34) and the tibial plateaus (22, 24), the degeneration of the support structures (ligaments) or bone on bone contact between the patella and the femur. Such damage or injury is usually derived from a degenerative disease, traumatic injury and/or aging. As the medial compartment usually transfers a larger portion of the load, damage or injury usually occurs in the medial TF compartment first, leaving the lateral compartment, at least initially, intact. This is illustrated in FIG. 2, where the medial femoral condyle (34) and the medial tibial plateau (24) makes bone-on-bone contact, while the lateral joint space (40) remains deeper than 5 mm, clearly showing that no degeneration has taken place. This situation will be confirmed by a stress view, shown in FIGS. 9 and 10. In FIG. 9, moments are applied about the knee joint, to result in a compressive force on the lateral TF compartment. Since no contact between the lateral femoral condyle (32) and the lateral tibial plateau (22) is present, with lateral joint space (40) remaining in excess of 5 mm deep, it is clear that the lateral TF compartment is unaffected by degeneration. In FIG. 10, opposite moments to those applied in FIG. 9 are applied about the knee joint, resulting in a compressive force in the medial TF compartment. Here, contact between the medial femoral condyle (34) and the medial tibial plateau (24) is indicative of medial TF degeneration. Obesity and genetic predisposition can aggravate the rate and severity of the degeneration. In cases where ligaments are compromised, subluxation, a situation where the femoral centreline (50) and the tibial centreline (60), no longer meet in the joint space or no longer line up, may occur. This is illustrated in FIGS. 5 and 8, where offset (42) indicates the subluxation. Subluxation represents a balance shift within the joint, and causes increased load on one of the compartments. In severe cases, the intercondylar eminence (not indicated) may even make contact with either the medial or lateral femoral condyle. The shape of the intercondylar eminence and particularly the sharp contact area causing a high stress concentration is such that contact with one of the femoral condyles might cause damage to the cartilage and increase pain.

In addition to the above, the knee joint (10) responds to the degeneration by the formation of osteophytes (not shown), which form in a natural process towards stabilization of a damaged knee joint (10). The presence of osteophytes, unfortunately also leads to additional discomfort and associated pain.

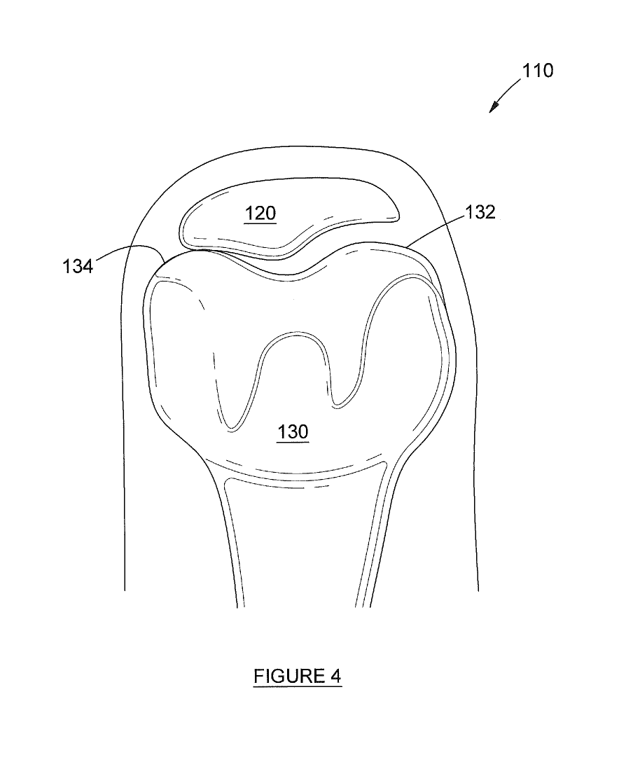

Another form of degeneration may be present in the form of a degenerated patellofemoral joint (PFJ). FIG. 4 shows a skyline view of a degenerated PFJ. Here it is clear that patella (120) makes bone-on-bone contact with the medial femoral condyle (134). Treatment of a severely degenerated PFJ can be in the form of a patellofemoral arthroplasty (PFA).

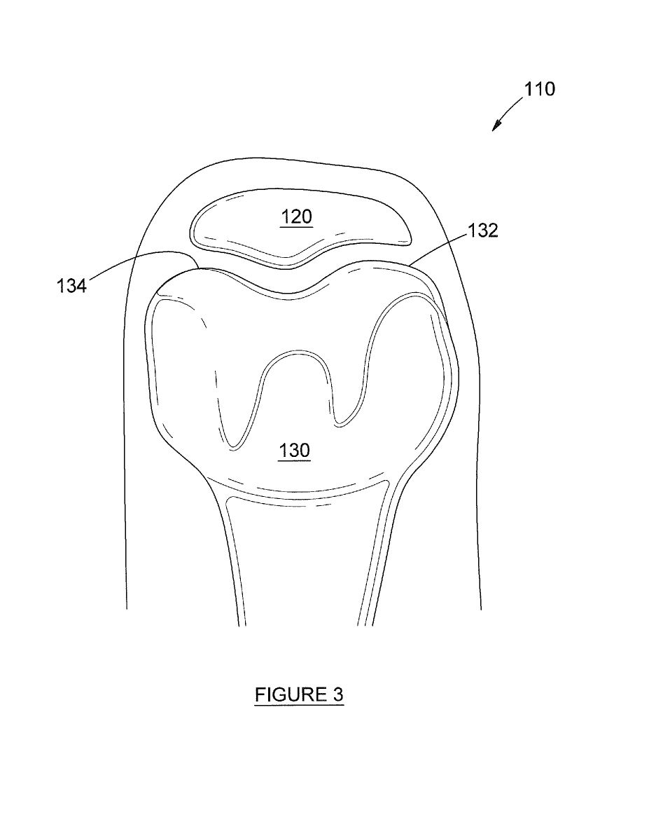

FIG. 3 shows a skyline view of the patella, with a knee joint typically indicated by reference numeral 110. The skyline view shows the anterior portion of the femur (130) with femoral condyles (132 and 134). The patella is indicated with reference numeral 120, and in FIG. 3, patella (120) is in its normal position. From this figure it is clear that in a normal knee (110) in skyline view, no contact between the patella (120) and the femur (130) is present.

When assessing the diseased or injured knee, an assessment instrument is utilized, which according to the current disclosure, is a radiographic image, and specifically an x-ray image. The embodiments of the present disclosure are not intended to replace existing methods of diagnosis, which includes a full medical history and clinical evaluation. The applicant has however found that, specifically in borderline cases, physicians are presented with uncertainty as to whether a PKA or a TKA would be a more appropriate form of treatment for a specific case. The disclosure is thus intended to augment patient selection criteria for either a PKA or a TKA, and to increase the likelihood of success in each treatment option.

The grading system criteria defines four grades according to which the extent of degeneration is determined once a diagnosis of a degenerative joint condition has been made, and whereby the suitable arthroplasty is prescribed by the grade of joint degeneration. The four grades indicated in the current embodiment of the disclosure are discussed in full below.

In general, the premise of the current method, as applied to TF PKAs lies in the proviso that two (the other TF and the PFJ) of the knee compartments has to be normal and unaffected by degeneration. This means that the depth of the joint spacing in the unaffected TF compartment, has to be at least 5 mm, which indicates that the chondral cartilage on the tibia and femur and meniscus is not excessively damaged or worn. The method further provides for cases where more than one compartment is affected, but where a TKA is not yet indicated. Here the single PKA will either be augmented by a second PKA or a PFA.

In all of the cases mentioned below, a wedge deformity in the “healthy” TF compartment (i.e. the joint spacing remains parallel but forms a converging angle in the healthy TF compartment) caused in a stress view, either indicates more wear or injury in the “healthy” TF compartment than initially anticipated or confirms the presence of ligamentous instability. In some cases, this might be a contraindication to the indicated grade of degeneration.

Grade 1:

Case 1: Single TF Compartment
Degeneration is confined to one TF compartment, with bone-on-bone degeneration present;
Osteophytes may be present in the other compartments, but these compartments generally retain their integrity;
The unaffected TF compartment must have a parallel joint, extending at least 5 mm deep (the meniscus and chondral cartilage must thus be unaffected and intact);
If a “wedge” sign is present, a PKA might not be suitable; and
Case 1 is illustrated in FIG. 2, where contact between medial femoral condyle (34) and medial tibial plateau (24) can be observed in the medial TF compartment. It is also clear that lateral joint space (40) remains parallel throughout the lateral TF compartment, and that the depth of the lateral joint space (40) remains in excess of 5 mm. No subluxation is present on the lateral view, indicative of the integrity of the ACL. The healthy TF compartment is further confirmed by the stress views of FIGS. 9 and 10, as discussed above.

Treatment: Partial Knee Arthroplasty (PKA)

Case 2: Only Patellar Femoral Joint involvement
TF compartments are unaffected;
The unaffected TF compartments will be confirmed by the 15 and 45 degree Rosenberg views and stress view similar to that illustrated in FIG. 1;
Osteophytes may be present in both the affected and healthy compartments, but these compartments must however retain their integrity;
Both TF compartments must have parallel joints, extending at least 5 mm deep (the meniscus and chondral cartilage must thus be unaffected and intact);
If a “wedge” sign in stress view is present, a patellofemoral arthroplasty (PFA) might not be suitable; and
Case 2 is illustrated in FIG. 4. From the skyline view of the compromised knee joint (110), it is clear that bone-on-bone contact between the patella (120) and the medial femoral condyle (134) is present.

Treatment: Patellofemoral Arthroplasty (PFA)

Grade 2:

Case 3: Pseudo-laxity confined to the TF compartments
Both of the TF compartments are affected with subluxation on the frontal plane;
Degeneration is limited to a single TF compartment, with minimal or no damage to the remaining TF compartment;
Ligaments, especially the ACL must be intact (pseudo-laxity) and the subluxation is due to large bone erosion defects, either lateral or medial (it is thus not damage to the ACL which causes the subluxation);
Extensive osteophytes will normally be present;
The stress view must indicate preservation of the healthy TF compartment with a parallel joint space throughout the width thereof, extending at least 5 mm, as is the case in FIG. 9;
“Wedge” sign in stress view of the unaffected TF compartment is a contraindication, as this proves that the ligaments are compromised or the healthy TF compartment is worn more severely than expected;
Pseudo-laxity of the ligaments is due to bony incongruency in the affected TF compartment;
This will be visible as subluxation on the anterior-posterior view, the extent of which will be reduced in the stress view;
A healthy contra-lateral compartment is confirmed with a parallel joint, extending at least 5 mm deep (the meniscus and chondral cartilage must thus be unaffected and intact), despite the possible presence of osteophytes; and
Case 3 is indicated in FIG. 5, where clear subluxation is present and defined by offset (42) between femoral centreline (50) and tibial centreline (60). Despite the bone-on-bone contact between the medial femoral condyle (34) and the medial tibial plateau (24), it is clear that the lateral TF compartment is unaffected and that a parallel lateral joint space (40) as well as a joint space (40) depth of at least 5 mm is present throughout the width of the lateral TF compartment.

Treatment: Partial Knee Arthroplasty (PKA)

Grade 3:

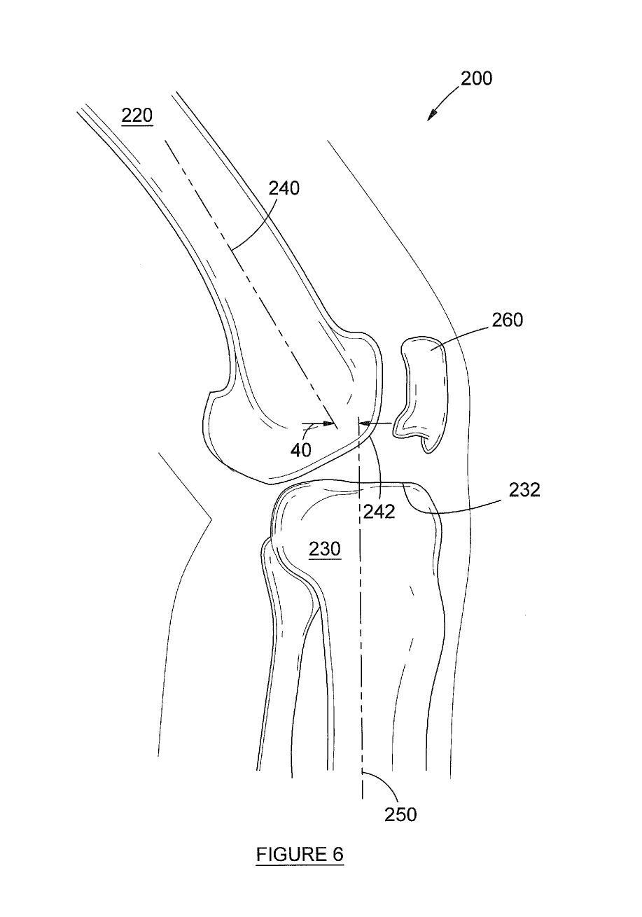

Case 4: Single TF compartment WITH concomitant Anterior Cruciate Ligament (ACL) instability
Isolated cruciate (ACL) ligamentous instability/deficiency or previous reconstruction;
FIG. 6 shows a schematic medial view where ACL instability is present. Anterior misalignment between the femur and the tibia is present in this instance; and
Osteoarthritis confined to a single TF compartment with a proven healthy contra-lateral TF compartment. The anterior view will be similar to that of FIGS. 2, 9 and 10.

Treatment: Anterior Cruciate Ligament (ACL) AND Partial Knee Arthroplasty (PKA)

Case 5: Single TF compartment WITH concomitant Patellar Femoral Joint Osteoarthritis
Grooved patellar femoral joint (PFJ) degeneration or subluxation, which is confirmed with a skyline view of the patella showing similar characteristics as that indicated in FIG. 4; and
Osteoarthritis confined to a single TF compartment.

Treatment: Patellar Femoral Joint (PFJ) AND Partial Knee Arthroplasty (PKA) or TKA

Grade 4:

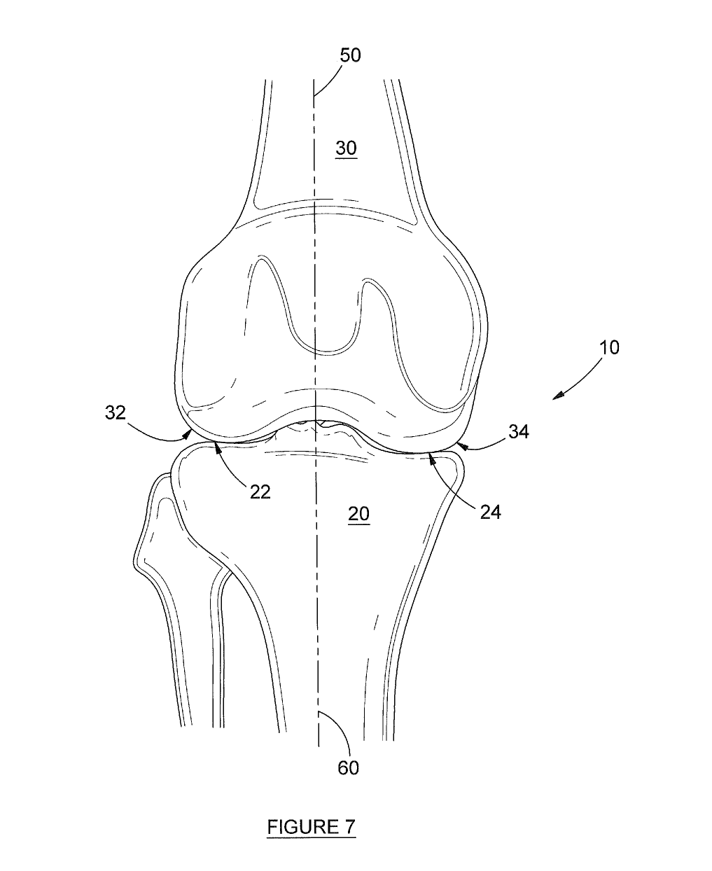

Case 6: Involvement of both TF compartments with ligamentous stability
Degeneration present in both TF compartments, i.e. less than 5 mm of joint space remains in both weight bearing compartments;
Ligaments remain stable and are not in need of mechanical, artificial replacement; and
Case 6 is illustrated in FIG. 7. Here it is clear that both the medial and lateral TF compartments are compromised, evidenced by the contact between lateral femoral condyle (32) and lateral tibial plateau (22) as well as medial femoral condyle (34) and medial tibial plateau (24). The stability of the support structures is evidenced from the lack of subluxation in the joint and confirmed on the stress views.

Treatment: Depending on the level of ligamentous stability
Bi-Condylar Partial Knee Arthroplasty (PKA) (ligaments are stable enough to support joint naturally); or
Bi-Cruciate retaining TKA or a Total Knee Arthroplasty (TKA).

Case 7: Involvement of both TF compartments with ligamentous instability
Degeneration present in both TF compartments, i.e. less than 5 mm of joint space remains in both TF compartments;
Ligaments are unstable and need a mechanical, artificial replacement; and
Case 7 is indicated in FIG. 8, where it is evident that, as in FIG. 7, bone-on-bone contact is present in both the medial and lateral TF compartments, with the additional subluxation indicated by offset (42), proving that the ACL is instable.

Treatment: Total Knee Arthroplasty (TKA)

In use, the surgeon will thus compare the abovementioned radiographic images of the patient to the predetermined criteria, thereby establishing which of the TF compartments are affected with bone-on-bone degeneration, whether damage to the patellar femoral joint is present and whether ligamentous instability is present.

Subsequently, the grade of degeneration of the knee joint will be established, and the suitable treatment option will be indicated.

Although only certain forms of the disclosure have been described herein, it will be appreciated by a person skilled in the art that other variations or modified versions of the current example are possible without departing from the spirit of the disclosure or the scope of this disclosure. For instance, the grading system does not have to be limited by the description provided here, and either a more simplified or more comprehensive grading system can be developed. Any such presentation or modification is therefore to be considered as falling within the spirit and scope of the present disclosure as described herein.

1. A method of determining a corrective surgical procedure for treatment of degeneration of a joint, including the steps of:
comparing a radiological image of the joint to a set of degenerative joint conditions to determine a diagnosis; and
correlating the diagnosis with a grading system which prescribes a corrective surgical procedure.

2. A method according to claim 1, wherein the joint is a knee joint.

3. A method according to claim 2, wherein the knee joint comprises a weight bearing joint, consisting of two opposing tibiofemoral (TF) compartments, and a patellofemoral (PF) joint.

4. A method according to claim 1, wherein the set of degenerative joint conditions comprises TF degeneration, PF degeneration, patellar grooving, subluxation of the weight bearing joint and anterior cruciate ligament (ACL) instability.

5. A method according to claim 1, wherein the corrective surgical procedure may be selected from the group consisting of a total knee arthroplasty (TKA), a partial knee arthroplasty (PKA), a patellofemoral joint arthroplasty (PFA) and an ACL reconstruction.

6. A method according to claim 1, wherein the grading system comprises a plurality of grades of joint degeneration based on the extent of degeneration of the joint.

7. A method according to claim 6, wherein a first grade is defined bydegeneration confined to either of the TF compartments, and wherein a PKA is indicated.

8. A method according to claim 6, wherein a first grade is defined by degeneration confined to the PF joint, and wherein a PFA is indicated.

9. A method according to claim 6, wherein a second grade is defined by degeneration confined to either of the TF compartments, resulting in subluxation across the weight bearing joint, and wherein a PKA is indicated.

10. A method according to claim 6, wherein a third grade is defined by degeneration confined to either of the TF compartments with concomitant ACL instability, and wherein ACL reconstruction and a PKA is indicated.

11. A method according to claim 6, wherein a third grade is defined by degeneration confined to either of the TF compartments with concomitant PF grooving, ACL instability or previous ACL reconstruction, and wherein a PFA and a PKA is indicated.

12. A method according to claim 6, wherein a fourth grade is defined by degeneration confined to both TF compartments, and wherein the corrective surgical procedure is selected from the group comprising bi-condylar PKAs and bi-cruciate retaining TKAs.

13. A method according to claim 6, wherein a fourth grade is defined by degeneration present in both TF compartments, with ligament instability resulting in subluxation across the weight bearing joint, and wherein a TKA is indicated.

14. A method according to claim 7, wherein a wedge deformity in an opposite TF compartment which is unaffected by degeneration is a contra-indication to the indicated corrective surgical procedure.

15. A method according to claim 1, wherein the radiological image is an x-ray image selected from the group of x-ray images of a knee joint consisting of a standing knee taken antero-posteriorly; a lateral view; a “stress-view” in 20° flexion; in 15° (medial) or 45° (lateral) postero-antero view (Rosenberg view); and a skyline view of the patella.
